(19) United States
(12) Patent Application Publication
(10) Pub. No.: US 2025/0295581 A1
(43) Pub. Date: Sep. 25, 2025

(54) Drug Releasing Hydrogels, Materials, Devices, and Uses Related Thereto

(71) Applicants: Emory University, Atlanta, GA (US); Georgia Tech Research Corporation, Atlanta, GA (US)

(72) Inventors: Adam M. Klein, Atlanta, GA (US); Daniel Refai, Atlanta, GA (US); Stephen W. Linderman, Atlanta, GA (US); Andres J. Garcia, Atlanta, GA (US)

(21) Appl. No.: 18/860,487

(22) PCT Filed: Apr. 27, 2023
(86) PCT No.: PCT/US2023/020145

§ 371 Date: Oct. 25, 2024

Related U.S. Application Data
(60) Provisional application No. 63/335,235, filed on Apr. 27, 2022.

(51) Int. Cl. A61K 9/00; A61K 47/34; A61L 27/18; A61L 27/52; A61L 27/54; A61L 27/58

(52) U.S. Cl. CPC A61K 9/0024; A61K 47/34; A61L 27/18; A61L 27/52; A61L 27/54; A61L 27/58; A61L 2300/402; A61L 2300/41; A61L 2300/604

(57) ABSTRACT

This disclosure relates to drug releasing implants. In certain embodiments, the implants release drugs from a biodegradable material. In certain embodiments, the implant is a low swelling hydrogel. In certain embodiments, the drug is an analgesic, an anti-inflammatory agent, anti-bacterial or other anti-microbial agent. In certain embodiments, this disclosure relates to methods of providing an implant as reported herein at a surgical site or site of injury for uses in reducing post operative pain and inflammation.

### CROSS-REFERENCE TO RELATED APPLICATIONS

This application claims the benefit of U.S. Provisional Application No. 63/335,235 filed Apr. 27, 2022. The entirety of this application is hereby incorporated by reference for all purposes.

### BACKGROUND

Anterior cervical discectomy and fusion (ACDF), sometimes referred to as an anterior cervical decompression, is a form of neck surgery near vertebrae in the neck that entails removing a damaged disc to relieve spinal cord or nerve root pressure which is accessed by an incision made in the front of the neck. An intravertebral disc is removed from between two vertebral bones. To stabilize the damaged segment, a bone graft and/or implants are fused in in the location where the intravertebral disc was removed. This surgery is common for patients with a painful cervical herniated disc, degenerative disc disease, or related conditions. Post-surgical difficulty swallowing (dysphagia) is common and can sometimes last for a prolonged period. Thus, there is a need to identify improved methods of addressing post-operative complications.

Haws et al. report data indicating that intraoperative steroid administration did not demonstrate an impact of local intraoperative steroid application on patient-reported swallowing function or swelling following ACDF. J Neurosurg Spine, 29:10-17, 2018.

Kim et al. report local administration of corticosteroids after multilevel ACDF can decrease postoperative severity and symptomatology of dysphagia. Spine, 2021, 46 (7): 413-420.

Truong et al. report nonswelling “Click” cross-linked gelatin and PEG hydrogels with tunable properties using pluronic linkers. Biomacromolecules, 2017, 18, 757-766.

Macdougall et al. report nonswelling thiol-yne cross-linked hydrogel materials as cytocompatible soft tissue scaffolds. Biomacromolecules, 2018, 19, 1378-1388.

Cidade et al. report injectable hydrogels based on pluronic/water systems filled with alginate microparticles for biomedical applications. Materials, 2019, 12, 1083.

Reference cited herein are not an admission of prior art.

### SUMMARY

This disclosure relates to drug releasing implants. In certain embodiments, the implant is a low swelling biodegradable hydrogel. In certain embodiments, the drug is an analgesic, an anti-inflammatory agent, anti-bacterial or other anti-microbial agent. In certain embodiments, this disclosure relates to methods of providing the biodegradable implant as reported herein at a surgical site or site of injury for uses in reducing post operative dysphagia, pain, and/or inflammation.

In certain embodiments, this disclosure relates to implants comprising a drug releasing low swelling hydrogel. In certain embodiments, the hydrogel comprises triblock copolymers comprising of two hydrophilic side chains and a central hydrophobic chain and terminal thiol groups which are crosslinked by a multi-arm linker.

In certain embodiments, the hydrogel is made by the process of providing the triblock copolymer in dimethylsulfoxide and contacting with the multi-arm linker in aqueous 4-(2-hydroxyethyl)-1-piperazineethanesulfonic acid (HEPES) buffer and a drug or polymer coated drug providing a cast hydrogel. In certain embodiments, alternative buffer solutions are contemplated. In certain embodiments, dimethyl sulfoxide is in an aqueous mixture. In certain embodiments, the dimethylsulfoxide is about 30% of the aqueous mixture by volume. In certain embodiments, the dimethylsulfoxide is about between 25% to 35% of the aqueous mixture by volume. In certain embodiments, the dimethylsulfoxide is greater than about 25%, 30% or 35% of the aqueous mixture by volume.

In certain embodiments, the hydrogel is made by the process of contacting the cast hydrogel with water providing a swollen hydrogel and freezing the swollen hydrogel providing a frozen swollen hydrogel.

In certain embodiments, the hydrogel is made by the process of contacting the frozen swollen hydrogel to an atmospheric pressure that is less than ambient atmospheric pressure providing a frozen swollen hydrogel at sub-atmospheric pressure

In certain embodiments, the hydrogel is made by the process of allowing the frozen swollen hydrogel at sufficient sub-atmospheric pressure to rise in temperature above freezing without substantial melting providing a lyophilized hydrogel.

In certain embodiments, the hydrogel absorbs water of more than 15- or 20-fold increase of weight compared to the weight of the lyophilized hydrogel. In certain embodiments, further contacting the lyophilized hydrogel with water provides a low swelling hydrogel that absorbs water of not more than 15-fold increase in weight compared to the lyophilized hydrogel.

In certain embodiments, contacting the lyophilized hydrogel with water is for more than one minute, more than one minute and less than 10 minutes before removing the source of water, or is for more than one minute and less than 30 minutes before removing the source of water.

In certain embodiments, the drug for release around the implanted area is a steroidal or non-steroidal anti-inflammatory agent embedded in particles. In certain embodiments, the drug is selected from dexamethasone, hydrocortisone, cortisone, prednisone, prednisolone, methylprednisolone, triamcinolone, betamethasone, or esters or salts thereof.

In certain embodiments, the hydrogel comprises triblock copolymers comprising of two hydrophilic side chains and a central hydrophobic chain and terminal thiol groups which are crosslinked by a multi-arm linker wherein the hydrophilic side chains are poly(ethylene oxide) and the hydrophobic chain is poly(propylene oxide).

In certain embodiments, the hydrogel comprises triblock copolymers comprising of two hydrophilic side chains and a central hydrophobic chain and terminal thiol groups which are crosslinked by a multi-arm linker wherein the hydrophilic side chains are poly(ethylene oxide) and the hydrophobic chain is poly(propylene oxide) wherein the polypropylene glycol segment has an average molecular weight of about 1800, and polyethylene glycol segments are about 40% of the total molecular weight.

In certain embodiments, the hydrogel comprises triblock copolymers comprising of two hydrophilic side chains and a central hydrophobic chain and terminal thiol groups which are crosslinked by a multi-arm linker wherein the triblock copolymers comprising of two hydrophilic side chains and a central hydrophobic chain and terminal thiol groups is a copolymer having the following formula:

—SCH₂CH₂(OCH₂CH₂)ₙ(OCH(CH₃)CH₂)ₘ(OCH₂CH₂)ₙOCH₂CH₂—S—

wherein the polypropylene glycol segment has an average molecular weight of about 1800, and polyethylene glycol segments are about 40% of the total molecular weight.

In certain embodiments, the multi-arm linker is a multi-arm polyethylene glycol maleimide based cross-linker. In certain embodiments, the multi-arm polyethylene glycol maleimide based cross-linker is C[CH₂O(CH₂CH₂O)ₚCH₂CH₂O(C═O)CH₂CH₂-(maleimide)]₄, C[CH₂O(CH₂CH₂O)ₚCH₂CH₂NH(C═O)CH₂CH₂-(maleimide)]₄, or combinations thereof.

In certain embodiments, the polyethylene glycol maleimide has an average molecular weight of about between 1 and 25 kDa.

In certain embodiments, the drug is embedded in particles with an average diameter of about between 0.1 to 100 micrometers which are embedded in the implant.

In certain embodiments, the drug is embedded in particles with an average diameter of about between 2 to 20 micrometers which are embedded in the implant.

In certain embodiments, the drug is embedded in particles with an average diameter of about between 2 to 5 micrometers which are embedded in the implant.

In certain embodiments, the drug is embedded in particles comprising poly(lactic acid) (PLA), poly(glycolic acid) (PGA), poly(lactic-co-glycolic acid) (PLGA) which are embedded in the implant.

In certain embodiments, biodegradable implant is in the form of a patch comprising a first side and a second side wherein the first side of the patch optionally contains an adhesive material for fixing the patch to bone, muscle, cartilage, tendon, collagen, or a metal or fixation device or material and the second side contains the anti-inflammatory agent.

In certain embodiments, the fixation device or material is a plate, screw, rod, pin, or suture,

In certain embodiments, biodegradable implant is in the form of a malleable or flexible patch.

In certain embodiments, the biodegradable implant is configured to fit on two vertebrae separated by a space represented by the thickness of an intervertebral disc in a human spinal column.

In certain embodiments, the biodegradable implant further comprising an anti-bacterial or anti-fungal agent.

In certain embodiments, this disclosure relates to methods of reducing post operative dysphagia after anterior cervical discectomy and fusion comprising implanting a biodegradable implant as disclosed herein on two cervical and/or thoracic vertebrae or device or material attached thereto.

### BRIEF DESCRIPTION OF THE SEVERAL VIEWS OF THE DRAWINGS

FIG. 1A illustrates and example of a biodegradable implant of this disclosure. Amphiphilic hydrogels for minimal swelling are loaded with drug-containing PLGA microparticles for sustained release. Illustrated is a bulk hydrogel wafer, which can be designed so one surface acts as an immobilization surface. However, it is contemplated that the immobilization surface is optional. The immobilization surface may be a biodegradable polymer that is sufficiently ridged so that one can pierce the surface with a fixation device, e.g., screw or suture, or it is contemplated that the immobilization surface is adhesive to surrounding bone or connective tissue or it is contemplated that the immobilization surface is adhesive to surrounding surgically implanted hardware.

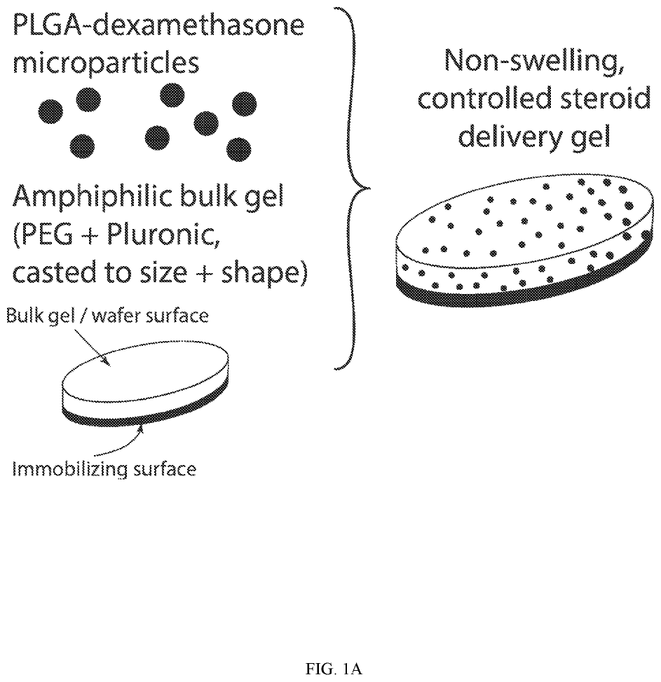

FIG. 1B shows additional view of a patch from a top and side angle.

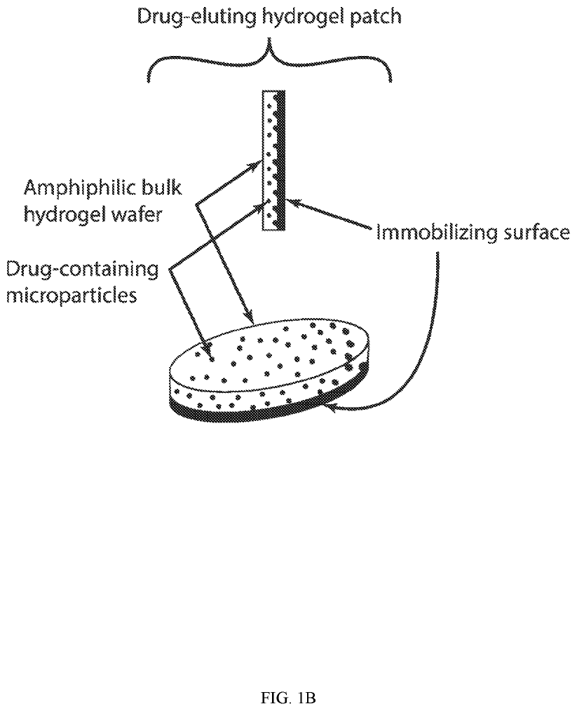

FIG. 2A illustrates implanting the biodegradable implant (patch) onto a vertebra (e). The patch can be fixed through an immobilization surface or by adhesion to a fixation device (hardware for spinal fusion). The patch is positioned on the front curved surface of the vertebra (e).

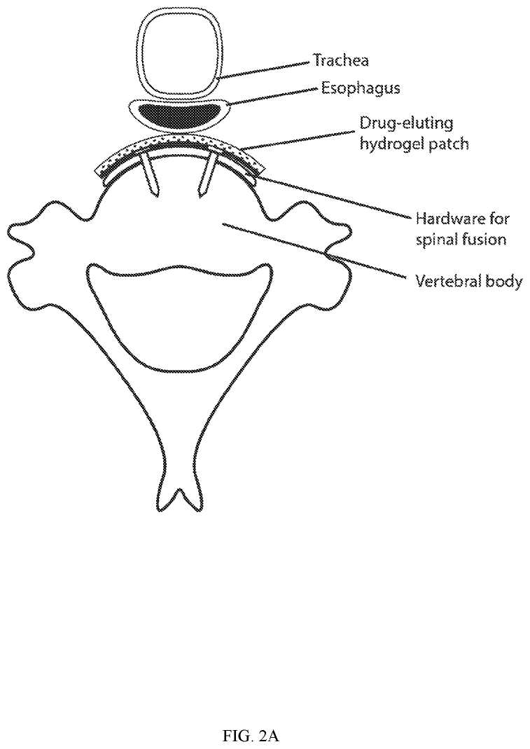

FIG. 2B illustrates a side view of the spine and vertebrae.

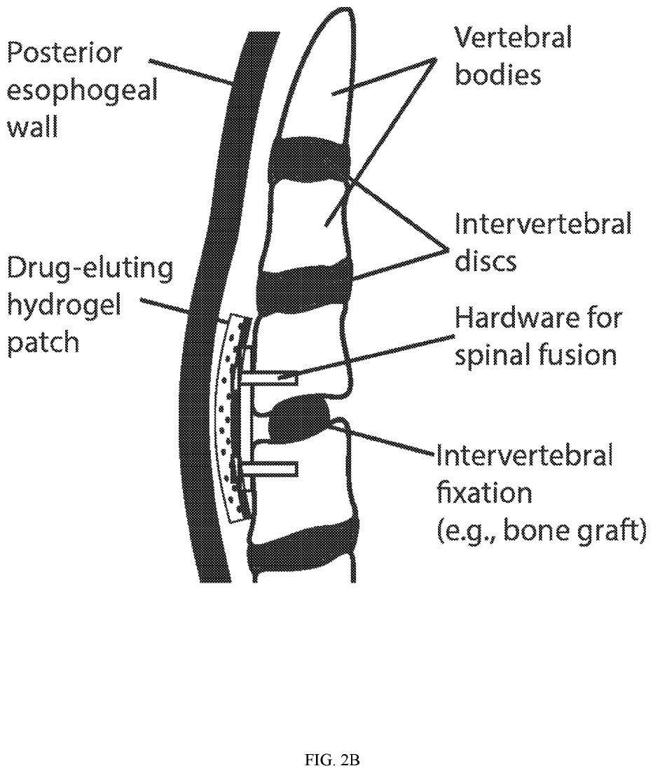

FIG. 3A illustrates the preparation of an example of the biodegrade implant. Poloxamer 184-dithiol (Pluronic™ L64-dithiol) crosslinker MW about 2900 g mol⁻¹, about 40% PEO: 60% PPO by weight and 4-arm PEG maleimide reacted with about 1:1 maleimide:thiol ratios. Triethylamine is an organic base that oxidizes thiol groups for reaction with maleimide groups by Michael addition chemistry. Titrated triethylamine (Et₃N) concentration (i.e., Et₃N:SH stoichiometric ratio) tunes reaction kinetics. Greater than 1:20 Et₃N:SH ratio crosslinks too quickly and is difficult to form consistent gel. About 1:30 Et₃N:SH stoichiometric ratio yields consistent crosslinking times. Less than 1:100 Et₃N:SH does not accelerate reaction enough to be useful.

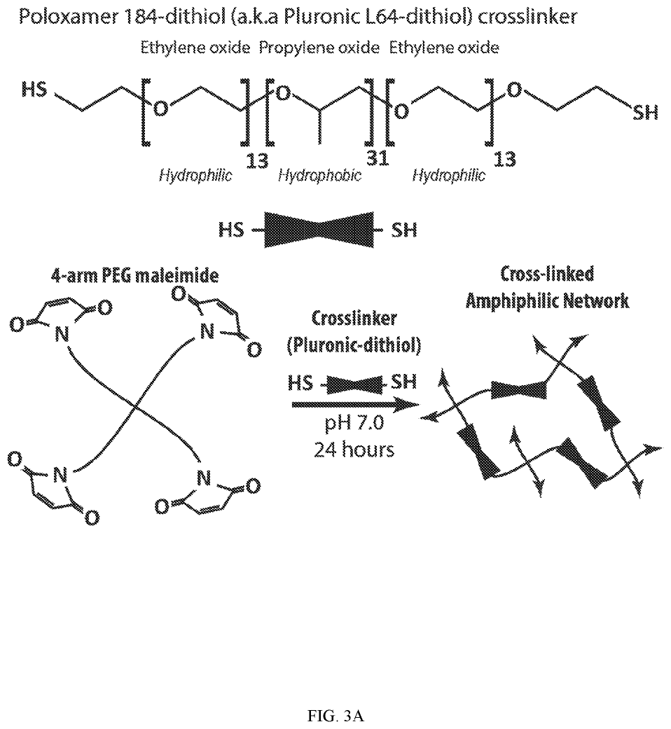

FIG. 3B shows data when preparing a low-swelling implant by lyophilizing the hydrogel after the initial preparation having different molecular weights, e.g., PEG-4 arm Maleimide at 5 kDa and 10 kDa crosslinked with modified poloxamer 184-dithiol (Pluronic™ L64-dithiol). Serial weights of amphiphilic PEG-4 arm Maleimide-Pluronic™ L64 dithiol gels, normalized by the lyophilized weight, under casting, swelling, and post-lyophilization reswelling conditions are shown. After lyophilization, gels swell to a substantially lower extent.

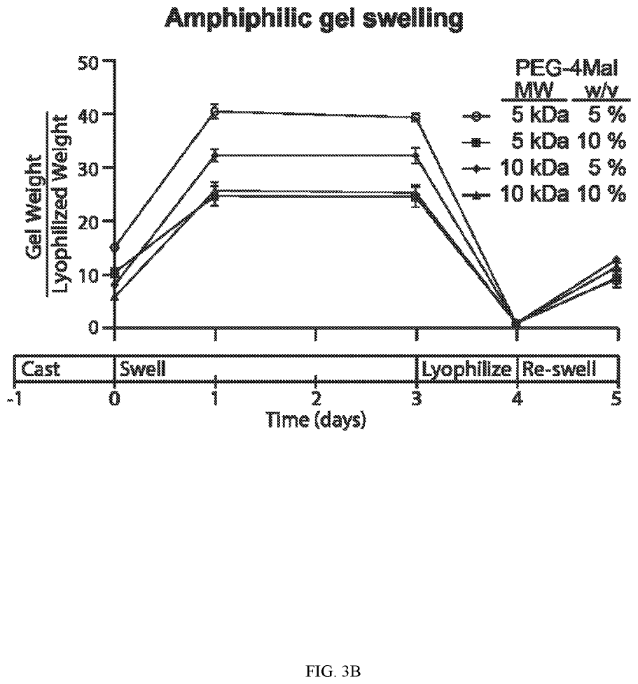

FIG. 3C shows data on storage modulus hydrogel using rheometry under different conditions. Rheometric properties represent amphiphilic gels in swollen states without lyophilization (A) and after lyophilization (B, C). Storage modulus increased 1 to 2 orders of magnitude from the swollen state to the lyophilized and reswollen state.

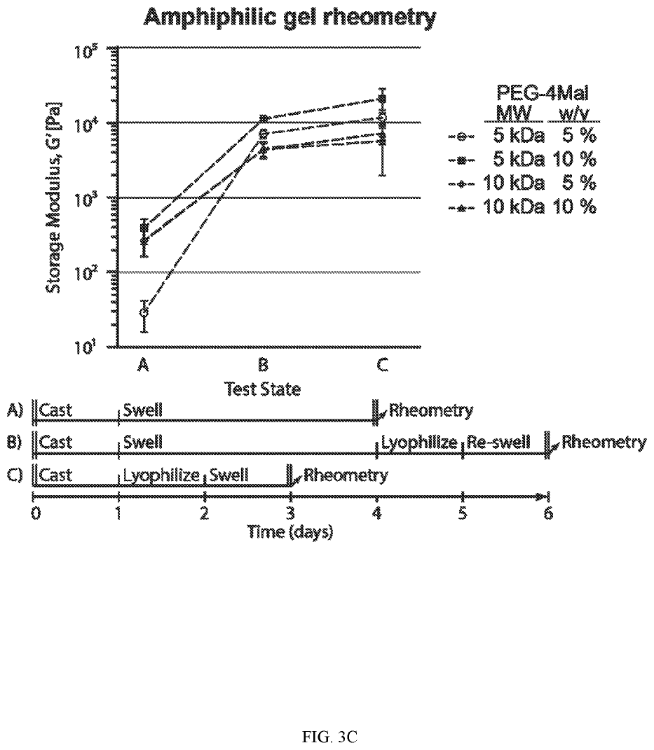

FIG. 4 shows data on reswelling rate of an amphiphilic gel after lyophilization, with serial weights over time in saline (n=4). The lyophilized weight, and the time 0 (immediately before reswelling and before contact with water or aqueous solution) the weights are identical, so the ratio is exactly 1. Hydrogels rapidly re-swell after lyophilization to a steady state weight within 5 minutes, allowing for intraoperative swelling.

FIG. 5A shows data on gel swelling with degradation in CAPS 50 mM at pH 10 using 4-arm polyethylene glycol amide or ester maleimide linkers.

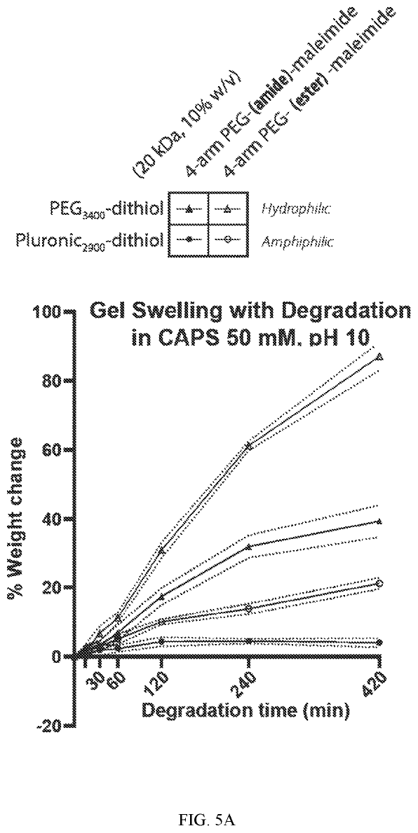

FIG. 5B shows gel rheometry data.

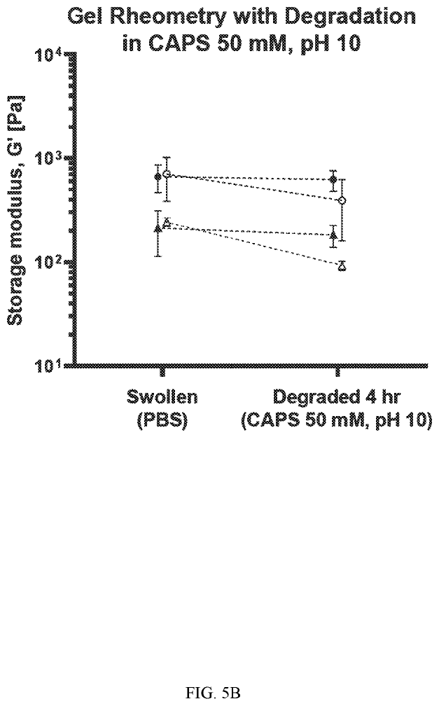

FIG. 5C shows size control of poly(lactic-co-glycolic acid) (PLGA)-dexamethasone microparticles based on homogenizer speed. During single oil-in-aqueous emulsion one is able to control PLGA-dexamethasone particle size in order to provided desirable drug release rates.

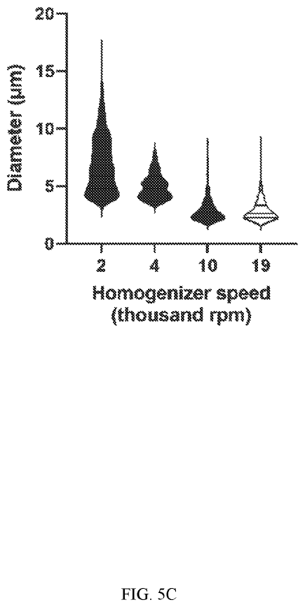

FIG. 6 shows data on cumulative dexamethasone release from amphiphilic (or hydrophilic control) hydrogels incorporating PLGA-dexamethasone microparticles. These drug-release curves demonstrate initial burst release followed by sustained release of the dexamethasone from the amphiphilic hydrogels disclosed herein.

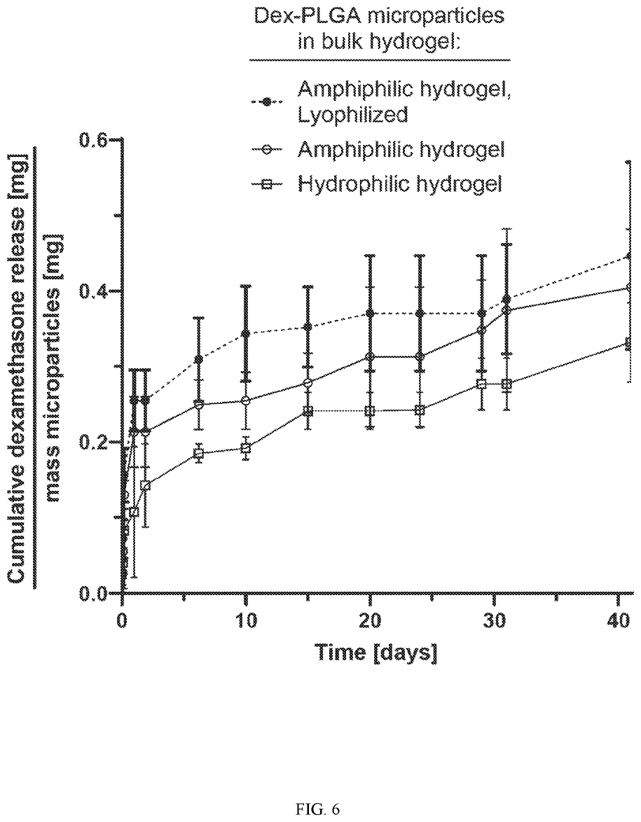

FIG. 7A shows a flowchart illustrating a method of using biodegradable patch constructs disclosed herein for anterior cervical discectomy and fusion.

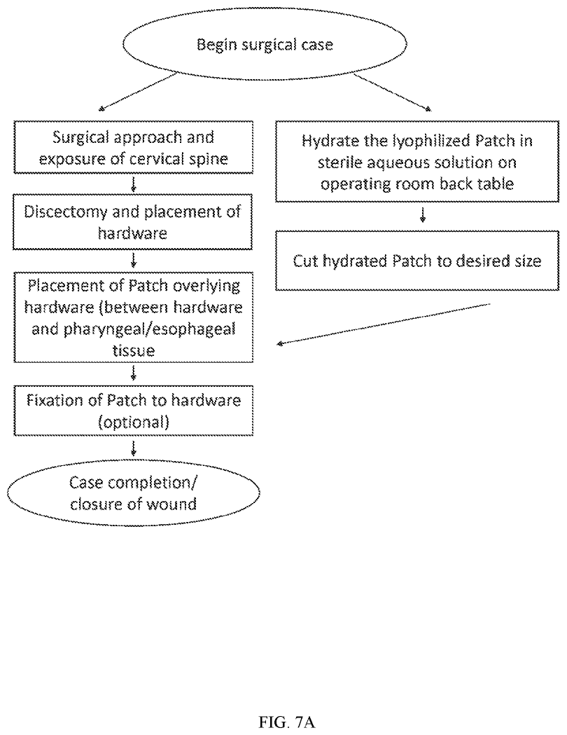

FIG. 7B shows a flowchart illustrating the creation of biodegradable patch constructs disclosed herein.

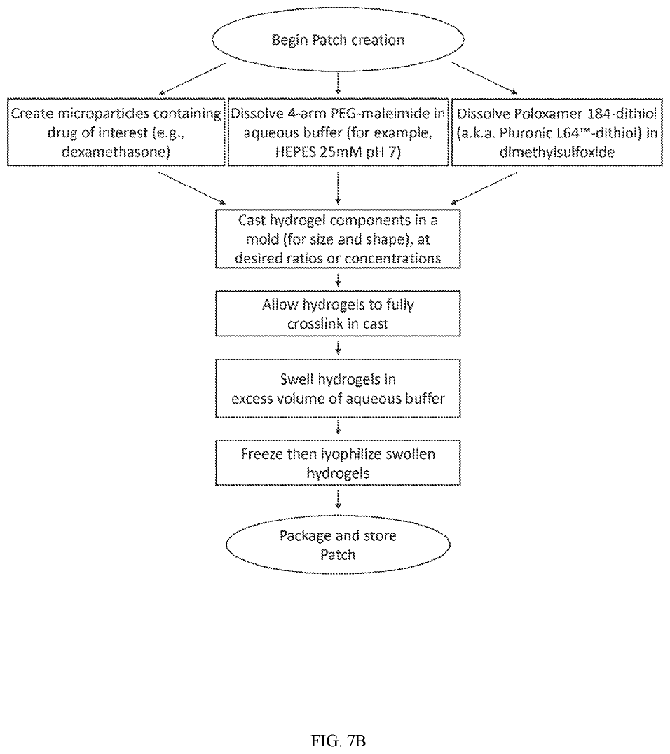

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims. Thus, reference to an “embodiment” refers to an example of the invention and is not necessarily limited by such an example.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

An “embodiment” of this disclosure refers to an example, but not necessarily limited to such example. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

The term “about” can include a difference of 5% or 10%.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

As used herein, “subject” refers to any animal, preferably a human patient, livestock, or domestic pet.

As used herein, the term “biodegradable” in reference to a material refers to a molecular arrangement in the material that when implanted to a subject, e.g., human, will be broken down by biological mechanism such that a decomposition of the molecular arrangement will occur and the molecular arrangement will not persist for over a long period of time, e.g., the molecular arrangement will be broken down by the body after a several days or a couple weeks. In certain embodiments, the disclosure contemplates that the biodegradable material will not exist after a month or several months.

The terms “drug,” “agent,” “pharmaceutical agent,” and similar terms are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect. Examples include analgesics, steroidal anti-inflammatories, non-steroidal anti-inflammatories, antibiotics, anti-bacterial agents, anti-neoplastics, anti-spasmodics, modulators of cell-extracellular matrix interactions, proteins, hormones, enzymes and enzyme inhibitors, anticoagulants and/or antithrombic agents, and vasodilating agents.

The terms “anti-inflammatory” and “anti-inflammatory agent” are also used interchangeably herein and mean and include a “pharmacological agent” and/or “active agent formulation”, which, when a therapeutically effective amount is present the drug prevents or treats bodily tissue inflammation i.e., the protective tissue response to injury or destruction of tissues, which serves to destroy, dilute, or wall off both the injurious agent and the injured tissues. Examples include alclofenac, alclometasone dipropionate, alpha amylase, amcinafal, amfenac sodium, anakinra, anirolac, balsalazide disodium, bendazac, benoxaprofen, bromelains, broperamole, budesonide, carprofen, cliprofen, clobetasol propionate, clobetasone butyrate, clopirac, cortodoxone, decanoate, deflazacort, depo-testosterone, desonide, desoximetasone, dexamethasone dipropionate, diclofenac potassium, diclofenac sodium, diflorasone diacetate, diflumidone sodium, diflunisal, difluprednate, dimethyl sulfoxide, enolicam sodium, etodolac, felbinac, fenamole, fenbufen, fenclofenac, fendosal, fenpipalone, fentiazac, flazalone, flufenamic acid, flunisolide acetate, flunixin, flunixin meglumine, fluorometholone acetate, flurbiprofen, fluticasone propionate, furaprofen, halcinonide, halobetasol propionate, ibuprofen, ibuprofen aluminum, ibuprofen piconol, indomethacin, indomethacin sodium, indoprofen, isoxepac, isoxicam, ketoprofen, lofemizole hydrochloride, loteprednol etabonate, meclofenamate sodium, meclofenamic acid, mefenamic acid, mesalamine, methenolone, methenolone acetate, nabumetone, nandrolone, naproxen, naproxen sodium, naproxol, olsalazine sodium, oxaprozin, oxyphenbutazone, oxymetholone, pirfenidone, piroxicam, piroxicam cinnamate, piroxicam olamine, pirprofen, proquazone, proxazole, proxazole citrate, salsalate, stanozolol, sudoxicam, sulindac, suprofen, talniflumate, tenidap, tenidap sodium, tenoxicam, testosterone, testosterone blends, tiopinac, tixocortol pivalate, tolmetin, tolmetin sodium, triclonide, triflumidate, zidometacin, and zomepirac sodium.

### Implants and Methods of Use

This disclosure relates to composition for releasing drugs from an implanted material. In certain embodiments, the implant is biodegradable or non-biodegradable. In certain embodiments, the implant is a low swelling hydrogel.

In certain embodiments, the drug is an analgesic, an anti-inflammatory agent, anti-bacterial or other anti-microbial agent. In certain embodiments, this disclosure relates to methods of providing the biodegradable implant as reported herein at a surgical site or site of injury for uses in reducing dysphagia, post operative pain, and inflammation.

In certain embodiments, the hydrogel contains triblock copolymers with two hydrophilic side chains and a central hydrophobic chain and terminal thiol groups which are crosslinked by a multi-arm linker. In certain embodiments, the multi-arm linker is a four-arm polyethylene glycol maleimide based cross-linker.

In certain embodiments, this disclosure contemplates making hydrogels disclosed herein by a method comprising contacting a triblock copolymer disclosed herein in an aqueous buffer, e.g., HEPES aqueous buffer, and optionally dimethylsulfoxide, and a four-arm maleimide based crosslinker disclosed herein forming cast hydrogels disclosed herein.

In certain embodiments, the hydrogel is made by the process of contacting the cast hydrogel with water providing a swollen hydrogel, freezing the swollen hydrogel providing a frozen swollen hydrogel, contacting the frozen swollen hydrogel to an atmospheric pressure that is less than ambient atmospheric pressure providing a frozen swollen hydrogel at sub-atmospheric pressure, allowing the frozen swollen hydrogel at sufficient sub-atmospheric pressure to rise in temperature above freezing without melting providing a lyophilized hydrogel. In certain embodiments, the atmospheric pressure that is less than ambient atmospheric pressure is between 5 and 13 pounds per square inch or less than 5 pounds per square inch.

In certain embodiments, the hydrogel initially absorbs water providing a swollen hydrogel of a more than 15- or 20-fold increases in weight compared to the weight of the lyophilized hydrogel. In certain embodiments, the method further comprises contacting the lyophilized hydrogel with water providing a low swelling hydrogel that absorbs water of not more than 5-, 10- or 15-fold increase weight when compared to the weight of the lyophilized hydrogel. In certain embodiments, further contacting the lyophilized hydrogel with water provides a low swelling hydrogel which is limited to between 8- to 15-fold increase in weight compared to the lyophilized hydrogel.

In certain embodiments, contacting the lyophilized hydrogel with water is for more than one minute, more than one minute and less than 10 minutes before removing the source of water, or is for more than one minute and less than 30 minutes before removing the source of water.

In certain embodiments, the analgesic or anti-inflammatory agent is a steroidal or non-steroidal anti-inflammatory agent. In certain embodiments, the drug is dexamethasone, hydrocortisone, cortisone, prednisone, prednisolone, methylprednisolone, triamcinolone, betamethasone, or esters or salts thereof.

In certain embodiments, the hydrophilic side chains are poly(ethylene oxide) and the hydrophobic chain is poly(propylene oxide). In certain embodiments, the triblock copolymers comprising of two hydrophilic side chains and a central hydrophobic chain and terminal thiol groups is a copolymer having the following formula:

—SCH₂CH₂(OCH₂CH₂)ₙ(OCH(CH₃)CH₂)ₘ(OCH₂CH₂)ₙOCH₂CH₂—S—.

In certain embodiments, the polypropylene glycol segment has an average molecular weight of about 1800, e.g., 1600-2000, and polyethylene glycol segments are about 40%, e.g., 35%-45% of the total molecular weight, e.g., wherein n is approximately 13, e.g., 12-15 and m is approximately 31, e.g., 29-33.

In certain embodiments, the hydrogel having triblock copolymers comprising of two hydrophilic side chains and a central hydrophobic chain and terminal thiol groups which are crosslinked by a multi-arm linker have an average molecular weight of about 2 kDa, e.g., between 1 and 3 kDa, or about 5 kDa, e.g., between 3 and 7 kDa, or about 10 kDa, e.g., between 8 and 15 kDa, or about 20 kDa, e.g., between 15 and 25 kDa.

In certain embodiments, the multi-arm linker is a multi-arm poly(ethylene glycol) maleimide based cross-linker. In certain embodiments, the multi-arm poly(ethylene glycol) maleimide based cross-linker is
C[CH₂O(CH₂CH₂O)ₚCH₂CH₂O(C═O) CH₂CH₂-(maleimide)]₄, (ester maleimide)
C[CH₂O(CH₂CH₂O)ₚCH₂CH₂NH(C═O)CH₂CH₂-(maleimide)]₄, (amide maleimide)
or combinations thereof.

In certain embodiments, the polyethylene glycol maleimide has an average molecular weight of about between 1 and 25 kDa. In certain embodiments, the polyethylene glycol maleimide has an average molecular weight of about 2 kDa, e.g., between 1 and 3 kDa, or about 5 kDa, e.g., between 3 and 7 kDa, or about 10 kDa, e.g., between 8 and 15 kDa, or about 20 kDa, e.g., between 15 and 25 kDa.

In certain embodiments, biodegradation rates are varied by including a variable ratio of 4-arm poly(ethylene glycol)-ester-maleimide to 4-arm poly(ethylene glycol)-amide-maleimide as the macromer that links the triblock copolymer. In certain embodiments, the ester to amide ratio is greater than 10:1, between 10:1 to 5:1, between 5:1 to 1:1, between 1:1 to 1:5; between 1:5 to 1:10, or greater than 1:10.

In certain embodiments, the drug is embedded in particles with an average diameter 0.1 to 100 micrometers which are embedded in the implant or wherein the drug is embedded in particles with an average diameter of 5 to 100 nanometers which are embedded in the implant.

In certain embodiments, it is contemplated that the microparticles have an average diameter of between 3 and 14 micrometers. In certain embodiments, it is contemplated that the microparticles have an average diameter of between 3 and 8 micrometers. In certain embodiments, it is contemplated that the microparticles have an average diameter of between 2 and 6 micrometers.

In certain embodiments, the drug is embedded in particles comprising poly(lactic acid) (PLA), poly(glycolic acid) (PGA), poly(lactic-co-glycolic acid) (PLGA) which are embedded in the implant.

In certain embodiments, the biodegradable implant is in the form of a patch comprising a first side and a second side wherein the first side/area of the patch optionally contains an adhesive material for fixing the patch to bone, muscle, cartilage, tendon, collagen, or a metal or fixation device or material and the second side/area contains the drug, analgesic, or anti-inflammatory agent.

In certain embodiments, the fixation device or material is a plate, screw, rod, pin, or suture,

In certain embodiments, the biodegradable implant is in the form of a malleable or flexible patch.

In certain embodiments, the biodegradable implant is configured to fit on two vertebrae separated by a space represented by the thickness of an intervertebral disc in a human spinal column. In certain embodiments, the biodegradable implant is lateral with the spine spanning the outer surface of a fixation device on the spine and over two vertebrae. In certain embodiments, the biodegradable implant is placed anterior to the vertebrae and posterior to the esophagus.

In certain embodiments, the biodegradable implant further comprising an anti-bacterial or anti-fungal agent.

In certain embodiments, this disclosure relates to methods of reducing post operative dysphagia after anterior cervical discectomy and fusion comprising implanting a biodegradable implant as in any of the claims herein on two cervical and/or thoracic vertebrae or device or material attached thereto.

In certain embodiments, the implants disclosed herein release drugs from a material implanted into a subject, e.g., human patient. In certain embodiments, thus disclosure relate to methods of reducing swelling, pain, and/or inflammation at a surgical site comprising,
incising the skin of a subject exposing a bone, muscle, tendon, cartilage, vertebra, intervertebral disc, organ, or other tissue or addressing exposed bone, vertebra, muscle, tendon, cartilage, vertebra, intervertebral disc, organ, or other tissue due to physical injury;
optionally removing the bone, vertebra, muscle, tendon, cartilage, vertebra, intervertebral disc, organ, or other tissue; or
optionally inserting and/or fixing a biocompatible device or biocompatible material or transplanted bone, vertebra, muscle, tendon, cartilage, vertebra, intervertebral disc, organ, or other tissue;
implanting a biodegradable implant effective for reducing post operative swelling, pain, and/or inflammation as disclosed herein in the area of the removed/exposed bone, muscle, organ, tendon, vertebra, intervertebral disc, organ, or other tissue or in the area of the inserted/fixed/transplanted bone, vertebra, muscle, tendon, cartilage, vertebra, intervertebral disc, organ, or other tissue or biocompatible device, or biocompatible material; and
optionally closing the surgical site by the use of sutures, staples, tissue glue, and/or adhesive tape.

### Steroid-Releasing, Low-Swelling Implant

In certain embodiments, this disclosure contemplates a hydrogel-based immediate and slow-release steroid delivery system designed for intraoperative application of topical steroid during surgery to minimize postoperative swelling, discomfort and/or related morbidity. One application is cervical spine surgery, which will minimize postoperative dysphagia and minimize the need for systemic steroids or narcotics.

Spine surgery is performed to address numerous spinal conditions and is typically conducted as an open procedure in which the affected spinal segment is exposed and fused for stability. One of the most common postoperative complications is dysphagia (swallowing difficulty), which can lead to significant postoperative morbidity and impact quality of life. This is sometimes due to edema (swelling) of the pharyngeal and esophageal tissue near the surgical site in the acute and subacute postoperative period. Traditionally, systemic steroids are employed to minimize the postoperative edema, but this introduces the systemic effects of oral steroids, as well as the challenge of swallowing pills in a situation where swallowing is difficult. It may also minimize the need for postoperative narcotic use.

In certain embodiments, disclosed herein is a hydrogel-based delivery system designed for intraoperative application of topical steroid during spine surgery with immediate and slow-release options in order to minimize postoperative morbidity, decrease the need for systemic steroids and/or narcotics. The biocompatible and biodegradable steroid-impregnated hydrogel is placed in a surgical field on the exposed cervical spine to lay between the spine and the esophageal and pharyngeal tissue.

### Immunomodulatory Drug Delivery Via Amphiphilic, Reduced-Swelling Hydrogels

Surgical interventions are frequently complicated by local inflammation and swelling, leading to postoperative pain, scarring, and foreign body responses. While hydrogels are effective vehicles for local, immunomodulatory drug delivery, extreme swelling upon degradation limits clinical applications. When implanted in tight surgical spaces (e.g., around the spine or airway), hydrogel swelling creates pressure on surrounding tissue that can cause pain and tissue necrosis. Amphiphilic hydrogel networks can minimize swelling. Here, the effects of thiolated PEG-PPG-PEG (Plu-DT) amphiphilic crosslinkers were evaluated versus hydrophilic thiolated PEG controls (PEG-DT) in PEG-4 arm maleimide (PEG-4Mal) networks on swelling and hydrogel stiffness with degradation. Compared to control hydrogels with similarly sized hydrophilic crosslinkers, amphiphilic gels exhibited lower swelling following casting. This effect was more pronounced in gels with lower PEG-4Mal molecular weight, i.e., higher hydrophobic content. Furthermore, amphiphilic hydrogels exhibited 2- to 3.5-fold lower swelling weights following lyophilization compared to before lyophilization, indicating significant fiber annealing beyond that seen in control hydrophilic hydrogels, possibly from clustering of hydrophobic segments. Within 7 hours of accelerated ester hydrolysis at pH 10, amphiphilic hydrogels (PEG-4-ester-Mal 20 kDa, 10% w/v, crosslinked with Plu-DT) swelled 21.3%±1.7% by weight, while hydrophilic hydrogels crosslinked with PEG-DT swelled 87.1%±3.4%, or 4.1-times more. After swelling to steady state, these amphiphilic hydrogels do not swell much further upon degradation. This is in contrast to most hydrogels, which do swell significantly with degradation and create pressure on surrounding tissue.

Finally, these amphiphilic hydrogels were loaded with dexamethasone containing PLGA microparticles for sustained drug release. Dexamethasone is a well-characterized anti-inflammatory drug. This approach allows for local, postoperative drug delivery for immune modulation in tight surgical spaces, with significantly reduced hydrogel swelling and reduced pressure on surrounding tissues. By reducing local tissue swelling and inflammation, these immunomodulatory drug delivery gels promise to reduce postoperative pain, scarring, and associated clinical complications.

### Amphiphilic Hydrogel Using 4-Arm PEG Maleimide Macromers

In order to overcome the swelling of typical bulk hydrogels as they degrade, which creates pressure on surrounding tissues, an in vitro crosslinking, amphiphilic bulk hydrogel was developed using 4-arm PEG maleimide macromers (PEG-4Mal) in HEPES buffer (5 kDa, 10 kDa, and 20 kDa molecular weights) crosslinked with an amphiphilic Pluronic™ L64-dithiol (60% wt PPG and 40 wt % PEG, average molar mass 2900 g mol⁻¹, approximately HS-PEG₁₃-PPG₃₁-PEG₁₃-SH) in dimethyl sulfoxide at 1:1 maleimide:thiol stoichiometric ratios. The hydrogels are first crosslinked in a cast/mold to a particular size and shape, at the conditions described herein. Following crosslinking, then the hydrogel is swelled and lyophilized.

For the poloxamer with the Pluronic™ tradename, coding the copolymers starts with a letter to define its physical form at room temperature (L=liquid, P=paste, F=flake (solid)) followed by two or three digits, The first digit (two digits in a three-digit number) in the numerical designation, multiplied by 300, indicates the approximate molecular weight of the central polymer e.g., PPG; and the last digit×10 gives the percentage of the polymer on the terminal ends (e.g., L64 indicates an average polypropylene glycol molecular mass of 1800 g/mol and a 40% polyethylene glycol content). Polymerization creates a distribution of products that have an average molecular distribution.

A range of crosslinking conditions were tested with different solvents for each component, varied mixing fractions, crosslinking timing, temperatures, and pH. Bulk gels yield consistent hydrogels at room temperature, pH 7, 70% aqueous PEG-4Mal+30% Pluronic™ L64-dithiol in DMSO, crosslinking for about 24-48 hours (16-72 hours wherein the exact time varies based on the PEG-4Mal molecular weight and the gel weight percentage). 5- and 10-kDa PEG-4Mal-poloxamer-dithiol gels crosslink fully at room temperature. PEG-4-Mal 20 kDa gels typically take longer than 48 hours to crosslink, and starting at room temperature initially followed by heating, e.g., at 37 C. The crosslinking time accelerates with inclusion of an organic base, triethylamine, in the DMSO phase to convert thiol groups (R—SH) to thiolate groups (R—S⁻), which accelerates the reaction with maleimide groups. A range of triethylamine concentrations were tested. Concentrations higher than 1:20 triethylamine:thiol stoichiometric ratio accelerated the reaction too much to get consistent crosslinking. Concentrations around 1:30 triethylamine:thiol yielded consistent hydrogel properties. Concentrations of triethylamine less than 1:100 triethylamine:thiol stoichiometric ratio did not accelerate the hydrogel polymerization reaction enough to be useful.

The crosslinking did not work in many of the conditions anticipated to be successful e.g., fully DMSO or EtOH solvent systems where all components are soluble. Using a combination of DMSO and an aqueous HEPES (4-(2-hydroxyethyl)-1-piperazineethanesulfonic acid) buffer was capable of forming the hydrogels in 16-72 hours. This extended crosslinking time is different from hydrogels made of PEG-4Mal with standard hydrophilic thiolated crosslinkers, which crosslink within seconds to minutes.

A swelling study of gels was performed with weights and rheometric properties measured directly after casting, after swelling 72 hours, or after lyophilizing then re-swelling. These showed consistent swelling and mechanical properties within gel types. Gel swelling extent is inversely related to PEG-4 Maleimide density (w/v %). Gels with 5 kDa PEG-4Mal were stiffer than gels with longer polymer arms (10 kDa), especially after lyophilization.

Lyophilization caused annealing of the bulk hydrogel network, resulting in gel stiffening and decreased swelling. For instance, 5 kDa PEG-4Mal-Pluronic™ L64 dithiol gels at 5% and 10% w/v swelled 40-fold and 24-fold compared to lyophilized weight, respectively. After lyophilization and reswelling, the same gels swelled to only 12- and 10-fold for 5% and 10% w/v, respectively. This is important because lyophilization will improve shelf storage life and reduce the risk of pressure on surrounding tissues from swelling.

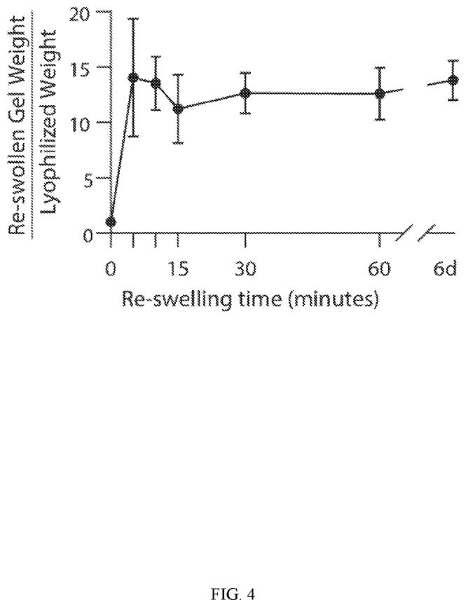

Reswelling occurs rapidly over 5-10 minutes (FIG. 4), indicating that lyophilized gels could be swollen in the operating room immediately prior to implantation to minimize the amount of additional swelling in the body. After initial swelling, there was no additional visible swelling of gels in PBS over a 3-week pilot study.

Pluronic L64-dithiol crosslinker makes amphiphilic hydrogels with decreased swelling and tunable mechanical properties based on PEG-4 Mal polymer size and weight percent. Lyophilization increases gel stiffness and further decreases swelling, possibly from annealing polymer arms and tighter clustering hydrophobic segments. Upon degradation, amphiphilic gels swell 4.1-fold to 9.6-fold less than hydrophilic gels. Degradation studies with lower PEG-4Mal polymer size (and therefore increased hydrophobic fraction by weight) are contemplated to further decrease swelling on degradation.

After swelling to steady state (e.g., after casting or after lyophilization), the amphiphilic hydrogels do not swell much more with degradation, i.e., limited swelling (e.g., less than 25% or 30%) after the initial wet gel casting. This is in contrast to hydrophilic hydrogels which swell large amounts on degradation. Experiments indicated that hydrogels disclosed herein swell less than comparable hydrophilic hydrogels on degradation, i.e., normalize to a steady state swelling size prior to degradation. Following swelling of the hydrogel to its steady state weight in aqueous solution (e.g., saline), the hydrogel does not swell more than 25-30% upon degradation. After 5 implanting in a subject, one may measure hydrogel weight or volume relative to the steady state swollen weight or volume. It is contemplated that after implantation of the stead state swollen hydrogel, one can use imaging methods, e.g., MRI and/or ultrasound, to detect the size, dimension, and/or volume. Volume dimensions can be used to calculate the weight of the implanted hydrogel, e.g., using a density approximation of about 1.0-1.1 g/mL.

TABLE 1
Experimental Results

| | Aqueous | | Ethanol | Isopropyl |
|---|---|---|---|---|
| Reagent | (HEPES 25 mM) | DMSO | (EtOH) | alcohol (IPA) |
| PEG-4Mal | Soluble | Soluble | Soluble | Insoluble |
| Plu2900-DT | Insoluble | Soluble | Soluble | Soluble |

| PEG-4Mal | Plu2900-DT | Crosslinking |
|---|---|---|
| buffer | buffer | extent (24 h) |
| HEPES (pH 7) | HEPES (pH 7) | Partial |
| HEPES (pH 5) | DMSO | None |
| HEPES (pH 7) | DMSO | Full |
| HEPES (pH 9) | DMSO | None |
| DMSO | DMSO | None |
| EtOH | EtOH | Partial |
| IPA | IPA | None |

| | PEG-4Mal | Plu2900-DT | Crosslinking |
|---|---|---|---|
| Temp | buffer (pH 7) | buffer | extent (24 h) |
| 4° C. | HEPES | DMSO | None |
| 22° C. | HEPES | DMSO | Full |
| 37° C. | HEPES | DMSO | Full |

Bold indicates extended crosslinking times. Crosslinking accelerates with inclusion of an organic base, e.g., triethylamine, with the Plu₂₉₀₀-DT

### Poly(Lactic-Co-Glycolic Acid) (PLGA) Microparticles Containing Dil (Indocarbocyanine Hydrophobic Fluorescent Dye)

PLGA microparticles degrade in pure dimethylsulfoxide (DMSO), and PLGA-dexamethasone microparticles degrade in pure DMSO. To prevent degradation, bulk gel production uses 30% DMSO in water. Experiments were performed to determine whether the microparticles would remain stable during bulk amphiphilic hydrogel production. Dil containing microparticles were placed in various DMSO/water solutions to check for degradation and Dil release, based on fluorescence measurements. At 48-hours, there is a similar, low amount of burst release between the negative control and the conditions with a mix of DMSO. DMSO (30% in water was used in bulk gel production) which does not degrade the microparticles.

What is claimed is:

1. A biodegradable implant comprising a drug releasing low swelling hydrogel;
wherein the drug is an analgesic or anti-inflammatory agent;
wherein the hydrogel comprises triblock copolymers comprising of two hydrophilic side chains and a central hydrophobic chain and terminal thiol groups which are crosslinked by a multi-arm linker; and
wherein the hydrogel is made by the process of providing the triblock copolymer in dimethylsulfoxide and mixing with the multi-arm linker in aqueous 4-(2-hydroxyethyl)-1-piperazineethanesulfonic acid (HEPES) buffer and the drug, providing a cast hydrogel;
contacting the cast hydrogel with water providing a swollen hydrogel;
freezing the swollen hydrogel providing a frozen swollen hydrogel;
contacting the frozen swollen hydrogel to an atmospheric pressure that is less than ambient atmospheric pressure providing a frozen swollen hydrogel at sub-atmospheric pressure; and
allowing the frozen swollen hydrogel at sufficient sub-atmospheric pressure to rise in temperature above freezing without melting providing a lyophilized hydrogel.

2. The biodegradable implant of claim 1, wherein the lyophilized hydrogel is contacted with and absorbs water providing a steady state swollen hydrogel.

3. The biodegradable implant of claim 2, wherein the steady state swollen hydrogel after implantation does not swell more than 30% by volume upon degrading.

4. The biodegradable implant of claim 2, wherein contacting the lyophilized hydrogel with water is for more than one minute and less than 30 minutes before removing the source of water.

5. The biodegradable implant of claim 1, wherein the drug is a steroidal or non-steroidal anti-inflammatory agent.

6. The biodegradable implant of claim 5, wherein the drug is dexamethasone, hydrocortisone, cortisone, prednisone, prednisolone, methylprednisolone, triamcinolone, betamethasone, or esters or salts thereof.

7. The biodegradable implant of claim 1, wherein the hydrophilic side chains are poly(ethylene oxide) and the hydrophobic chain is poly(propylene oxide).

8. The biodegradable implant of claim 1, wherein the triblock copolymers comprising of two hydrophilic side chains and a central hydrophobic chain and terminal thiol groups is a copolymer having the following formula:
—SCH₂CH₂(OCH₂CH₂)ₙ(OCH(CH₃)CH₂)ₘ(OCH₂CH₂)ₙOCH₂CH₂—S—
wherein the polypropylene glycol segment has an average molecular weight 1800, and polyethylene glycol segments are about 40% of the total molecular weight.

9. The biodegradable implant of claim 1, wherein the multi-arm linker is a multi-arm poly(ethylene glycol) maleimide based cross-linker.

10. The biodegradable implant of claim 9, wherein the multi-arm poly(ethylene glycol) maleimide based cross-linker is
C[CH₂O(CH₂CH₂O)ₚCH₂CH₂O(C═O)CH₂CH₂-(maleimide)]4,
C[CH₂O(CH₂CH₂O)ₚCH₂CH₂NH(C═O)CH₂CH₂-(maleimide)]4,
or combinations thereof.

11. The biodegradable implant of claim 10, wherein the polyethylene glycol maleimide has an average molecular weight of about between 1 and 25 kDa.

12. The biodegradable implant of claim 1, wherein the drug is embedded in particles with an average diameter 0.1 to 100 micrometers which are embedded in the implant.

13. The biodegradable implant of claim 1, wherein the drug is embedded in particles comprising poly(lactic acid) (PLA), poly(glycolic acid) (PGA), poly(lactic-co-glycolic acid) (PLGA) which are embedded in the implant.

14. The biodegradable implant of claim 1 in the form of a patch comprising a first side and a second side wherein the first side of the patch optionally contains an adhesive material for fixing the patch to bone, muscle, cartilage, tendon, collagen, or a metal or fixation device or material and the second side contains the anti-inflammatory or analgesic agent.

15. The biodegradable implant of claim 14, wherein the fixation device or material is a plate, screw, rod, pin, or suture,

16. The biodegradable implant of claim 14 in the form of a malleable or flexible patch.

17. The biodegradable implant of claim 14 configured to fit on two or more vertebrae separated by a space represented by the thickness of an intervertebral disc in a human spinal column.

18. The biodegradable implant of claim 14, further comprising an anti-bacterial or anti-fungal agent.

19. A method of reducing post operative dysphagia after anterior cervical discectomy and fusion comprising implanting a biodegradable implant of claim 1 on two or more vertebrae or device or material attached thereto.

20. A method of reducing swelling, pain, and/or inflammation at a surgical site comprising,
incising the skin of a subject exposing a bone, muscle, tendon, cartilage, vertebra, intervertebral disc, organ, or other tissue or addressing exposed bone, vertebra, muscle, tendon, cartilage, vertebra, intervertebral disc, organ, or other tissue due to physical injury;
optionally removing the bone, vertebra, muscle, tendon, cartilage, vertebra, intervertebral disc, organ, or other tissue; or
optionally inserting and/or fixing a biocompatible device or biocompatible material or transplanted bone, vertebra, muscle, tendon, cartilage, vertebra, intervertebral disc, organ, or other tissue;
implanting a biodegradable implant of claim 1 effective for reducing post operative swelling, pain, and/or inflammation in the area of the removed/exposed bone, muscle, organ, tendon, vertebra, intervertebral disc, organ, or other tissue or in the area of the inserted/fixed/transplanted bone, vertebra, muscle, tendon, cartilage, vertebra, intervertebral disc, organ, or other tissue or biocompatible device, or biocompatible material; and
optionally closing the surgical site by the use of sutures, staples, tissue glue, and/or adhesive tape.
